(19) United States
(12) Patent Application Publication
(10) Pub. No.: US 2026/0256975 A1
(43) Pub. Date: Sep. 3, 2026

(54) ODOR REMOVAL SYSTEM AND METHOD

(71) Applicant: Odor Control Systems LLC, Spring, TX (US)

(72) Inventors: BLAINE JOSEPH, THE WOODLANDS, TX (US); SLADE SWANNER, SPRING, TX (US); DAVID MENDEZ, FRIENDSWOOD, TX (US)

(21) Appl. No.: 19/264,833

(22) Filed: Jul. 9, 2025

Related U.S. Application Data
(60) Provisional application No. 63/765,986, filed on Mar. 3, 2025.

(51) Int. Cl. A61L 9/14; A61L 9/015; A61L 101/28

(52) U.S. Cl. CPC A61L 9/145; A61L 9/015; A61L 2101/28; A61L 2209/11; A61L 2209/13

(57) ABSTRACT

A system and method for removing odors from wastewater facilities, including wastewater lift stations and wastewater plants, and other locations where organic material decomposes. In a first embodiment, a liquid contact tank is filled with an iron chelating liquid and is coupled to a gas inlet pipe attached to the source of the odors. The odorous gas is drawn by a blower/compressor through a submerged aerator located in the liquid contact tank, where the gas interacts with the iron chelating liquid to remove the odor-causing molecules. The residual gas is vented out of the top of the liquid contact tank to the atmosphere. The iron chelating liquid is continuously circulated by an inline pump. Both the pump and the blower/compressor are installed with a variable frequency drives allowing the devices to run at an optimal speed.

### PRIORITY CLAIM AND CROSS REFERENCE

This application claims priority to U.S. Provisional Patent Application No. 63/765,986; filed Mar. 3, 2025 and entitled “Odor Removal System and Method,” which application is hereby incorporated by reference in its entirety.

### BACKGROUND

Wastewater facilities generally produce nuisance odors due to the decomposition of organic material. Specifically, these odors are caused by the presence of reduced sulfur compounds, such as hydrogen sulfide. This disclosure relates generally to removing these odors from wastewater lift stations and wastewater facilities and specifically to a method and system for removing reduced sulfur compounds, such as hydrogen sulfide from a gas supply originating from various wastewater facilities.

The presence of odor at wastewater facilities is the cause of one of the most frequent complaints by wastewater facility customers due to the proximity of the wastewater facility to customer homes. Additionally, due to the nature of reduced sulfur compounds, such as hydrogen sulfide, small quantities of reduced sulfur compounds in the atmosphere cause significant foul smelling odors.

Odor removal systems using solid odor absorbing media are generally used at wastewater facilities. A typical odor removal system uses an expensive charcoal based system that absorbs odors. This solid media requires significant manual labor and expense to dispose of the consumed media correctly. Due to the frequent complaints from wastewater facility customers, removing odors from the wastewater facilities is a top priority.

A method for producing and using alkaline aqueous ferric iron solutions to remove hydrogen sulfide (H₂S) and other reduced sulfur compounds from fluids, including gases and liquids, is known in the art. The method involves contacting the sulfuric fluid with an alkaline aqueous solution containing a selected concentration of ferric ion, Fe(III), where the ferric ion is dissolved in the aqueous medium. Contact results in capture and oxidation of at least a portion of the reduced sulfur compounds in the fluid, the concomitant formation of ferrous ion, Fe(II), the formation of suspended Fe(II) sulfide particles, and removal of reduced sulfur compounds from the fluid. After contact with the fluid, the alkaline aqueous solution used to remove reduced sulfur and the ferrous sulfide particles suspended therein can be regenerated by treatment with oxygen in air or an alternative oxidizing agent, which results in formation of elemental sulfur that precipitates from the solution.

U.S. Pat. No. 11,021,375, entitled “Methods for producing and using alkaline aqueous ferric iron solutions” and issued Jun. 1, 2021, discloses such a method, and is incorporated herein by reference in its entirety. The system disclosed in the '375 patent is a relatively complex system suitable to remove hydrogen sulfide in large industrial processes. Due to its complexity, size, and large capital cost, the '375 system is not suitable for local wastewater facilities.

A primary object of one or more embodiments is to minimize the capital cost of removing odors from wastewater facilities.

Another object of the system is to provide a method which minimizes the operational costs of the wastewater facilities by providing a method that requires little intervention and involvement.

Another object of the system is to provide a method that reduces neighborhood complaints about odors due to the improved efficacy of using a liquid based media.

### BRIEF DESCRIPTION OF THE DRAWINGS

Many aspects of the present disclosure can be better understood with reference to the following drawings, in which:

FIG. 1 is a piping and instrumentation diagram of an odor removal system according to one or more embodiments.

The components in the drawings are not necessarily to scale, with emphasis instead being placed upon clearly illustrating the principles of the disclosure. Moreover, in the drawings, like reference numerals designate corresponding parts throughout the several views. Terms of orientation, such as top, bottom, front, back, left, and right, are used herein to aid the reader in understanding the disclosure. These terms are not intended to be limiting, and the described orientation may be changed without limiting the scope of the disclosure.

### DETAILED DESCRIPTION

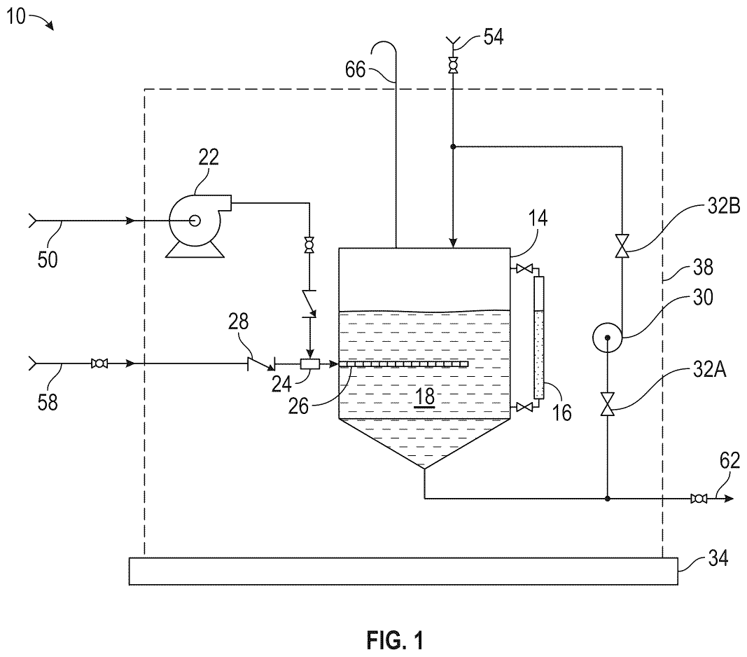

The objects identified above, as well as other features of the invention are incorporated in a system and method for removing gaseous odors from wastewater facilities. FIG. 1 is a piping and instrumentation diagram of an odor removal system 10 according to one or more embodiments.

Referring to FIG. 1, in a preferred embodiment, odor removal system 10 has a small footprint and may be housed on a pallet, skid or small slab 34 and enclosed by an enclosure 38, which may include one or more hatches, access plates, covers, windows, or doors for ready access to system 10 as may be required for installation, operation, and maintenance or repair. Odor removal system 10 includes a fresh air supply inlet 50, a fill inlet 54, a wastewater gas inlet 58, a drain outlet 62, and a vent 66.

Odor removal system 10 includes a liquid contact tank or vessel 14 for holding a liquid iron chelating media 18, such as that disclosed in the '375 patent, incorporated herein. Tank 14 is fluidly coupled to fill inlet 54 at or near the top of the vessel through which chelating media 18 is introduced into the tank. Tank 14 is fluidly coupled to drain outlet 62 at or near the bottom of the vessel by which tank 14 may be drained for maintenance purposes. Tank 14 is also fluidly coupled at or near the top of the vessel to vent 66 for equalizing pressure and exhausting gases introduced into tank 14 during operation of the system, as described hereinafter.

In order to eliminate possible stagnation of the iron chelating media 18, odor removal system 10 includes a pump 30, shown in FIG. 1 being fluidly coupled between the drain outlet 62 line and the fill inlet 54 line, circulates the liquid iron chelating media 18. Pump 30 is illustrated as a small centrifugal pump, but other suitable pumps may be used, as will be understood by a routineer in the art. Isolation valves 32A, 32B are provided so that pump 30 may be easily removed for maintenance or repair. Although pump 30 is shown as coupled to the drain outlet and fill inlet lines, one of ordinary skill in the art will recognize that pump 30 may be fluidly coupled to tank 14 via dedicated lines if desired.

Pump 30 is ideally operable from single phase mains voltage—120 or 240 volts in the United States. However, three phase and/or high voltage motors may be used where appropriate. According to one or more embodiments, pump 30 may have a variable frequency drive to allow efficient adjustment of the circulation flow rate. In alternative embodiments, pump 30 may be controlled by a timer, or a throttle valve or recirculation valve may be provided to reduce the circulation flow rate if necessary.

Ideally, a sight glass 16 is fluidly isolably connected to tank 14 to allow indication of the fluid level within the vessel, as is known to routineers in the art. Preferably, sight glass 16 is located outside of enclosure 38 for easy viewing, or enclosure 38 has a window or other means for easy viewing of sight glass 16 if located with the enclosure. However, other means or instrumentation for providing fluid level within tank 14 may be used as known in the art.

The exterior end of wastewater gas inlet 58 is fluidly connected to a vent (not illustrated) of the wastewater treatment facility or lift station. System 10 further includes an air blower/compressor 22 that is operable to draw wastewater gas into tank 14 by Bernoulli effect. The inlet of blower 22 is fluidly coupled to fresh air supply inlet 50. Although not illustrated, a screen or filter may be provided to prevent, insects, leaves, and other particulate matter from being ingested into blower 22. The outlet of blower 22 is fluidly coupled to a jet pump or venturi generator 24, which may be an eductor, injector, or in the most simple case, a tee fitting. The interior end of wastewater gas inlet 58 is fluidly connected to venturi generator 24 via a check valve 28, which serves to prevent blower 22 from blowing air into the wastewater treatment facility or lift station. The output of venturi generator 24 is fluidly connected to an aerator or sparging device 26, which is disposed within tank 14 at a position near the bottom of the vessel and below a volume of liquid iron chelating media 18.

Blower 22 is ideally operable from single phase mains voltage—120 or 240 volts in the United States. However, three phase and/or high voltage motors may be used where appropriate. According to one or more embodiments, blower 22 may have a variable frequency drive to allow efficient adjustment of the circulation flow rate. In alternative embodiments, a throttle valve or recirculation valve may be provided to reduce the blower flow rate if necessary.

In operation, the sulfur compound-laden gases from wastewater facility are introduced into the odor removal system 10 by a vacuum being created by blower 22/venturi generator 24 combination, thereby forcing the air/wastewater gas mixture facility into tank 14 via sparging device 26. Hydrogen sulfide and other sulfur compounds then interact with the iron chelating media, which absorbs the sulfur compounds and removes the odor from the gas. Excess sulfide-free gas exits tank 14 via vent 66 into the atmosphere. The sulfur compounds are continually separated from the iron chelating media 18 in the presence of oxygen from sparging device 26. Nevertheless, during operation, the sulfur compound concentration should be regularly tested to verify the efficacy of the iron chelating media 18.

Although the system and method described herein is particularly for wastewater facilities, it may find equal applicability in any application where decomposition of organic material causes sulfide-based unwanted odors.

The Abstract of the disclosure is solely for providing a way by which to determine quickly from a cursory reading the nature and gist of technical disclosure, and it represents solely one or more embodiments.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. An odor removal system comprising:
a liquid contact tank for containing an iron chelating liquid;
a sparging device disposed within said tank;
a means for introducing an odorous gas containing sulfur compounds into said tank through said sparging device; and
a vent fluidly coupled to said tank.

2. The odor removal system of claim 1, further comprising:
a volume of said iron chelating liquid disposed within said tank and covering said sparging device; whereby
said sulfur compounds interacting with the volume of iron chelating liquid, which absorbs the sulfur compounds and removes odor from the gas.

3. The odor removal system of claim 1, wherein said means for introducing an odorous gas comprises:
a venturi generator having first and second inlet ports and an outlet port;
a blower having an outlet fluidly coupled to the second inlet port of said venturi generator; and
the sparging device being fluidly coupled to the outlet port of said venturi generator; whereby
a source of said odorous gas may be fluidly coupled to the first inlet port of said venturi generator; and
operation of the blower creates a suction at the first port of the venturi generator and is operable to draw the odorous gas into the tank via the sparging device.

4. The odor removal system of claim 3, further comprising:
a variable frequency drive operatively coupled to said blower.

5. The odor removal system of claim 1, further comprising:
a circulation pump having an inlet and an outlet;
the inlet of the circulation pump being fluidly coupled to the tank at or near the bottom of the tank; and
the outlet of the circulation pump being fluidly coupled to the tank; whereby operation of said circulation pump prevents stagnation of said iron chelating liquid.

6. The odor removal system of claim 5, further comprising:
a variable frequency drive operatively coupled to said pump.

7. An odor removal system comprising:
a liquid contact tank;
a volume of said iron chelating liquid disposed within said tank;
a sparging device disposed within said tank and within said volume of said iron chelating liquid;
a venturi generator having first and second inlet ports and an outlet port, the sparging device being fluidly coupled to the outlet port of said venturi generator;
a blower having an outlet fluidly coupled to the second inlet port of said venturi generator;
a circulation pump having an inlet and an outlet, the inlet of the circulation pump being fluidly coupled to the tank at or near the bottom of the tank and the outlet of the circulation pump being fluidly coupled to the tank; whereby
a source of said odorous gas may be fluidly coupled to the first inlet port of said venturi generator;
operation of the blower creates a suction at the first port of the venturi generator and is operable to draw the odorous gas into the tank via the sparging device; and
operation of said circulation pump prevents stagnation of said iron chelating liquid.

8. The odor removal system of claim 7, further comprising:
a first variable frequency drive operatively coupled to said blower; and
a second variable frequency drive operatively coupled to said pump.

9. The odor removal system of claim 7, further comprising:
a skid carrying said tank, said pump, said blower, and said venturi generator; and
an enclosure carried by said skid and housing said tank, said pump, said blower, and said venturi generator.

10. A method for removing odors from a gas containing sulfur compounds, comprising:
providing a liquid contact tank having a sparging device disposed therein;
filling a volume of iron chelating liquid within said tank so as to cover said sparging device with said liquid; and
introducing said gas containing sulfur compounds into said iron chelating liquid through said sparging device; whereby
said sulfur compounds interacting with the volume of iron chelating liquid, which absorbs the sulfur compounds and removes odor from the gas.

11. The method of claim 10, further comprising:
fluidly coupling said sparging device to an outlet of a venturi generator;
fluidly coupling a source of said gas containing sulfur compounds to a first inlet of said venturi generator; and
introducing a pressurized stream of air into a second inlet of said venturi generator; whereby
said pressurized stream of air draws said gas containing sulfur compounds into said venturi generator and into said iron chelating liquid through said sparging device.

12. The method of claim 11, further comprising:
fluidly coupling an outlet of a blower to said second inlet of said venturi generator; whereby
said blower introduces said pressurized stream of air.

13. The method of claim 13, further comprising:
controlling the rate of said introduction of pressurized stream of air.

14. The method of claim 13, further comprising:
controlling the speed of said blower with a variable frequency drive.

15. The method of claim 10, further comprising:
circulating said volume of iron chelating liquid within said tank.

16. The method of claim 15, further comprising:
fluidly coupling an inlet of a circulation pump to the tank at or near the bottom of the tank; and
fluidly coupling an outlet of the circulation pump to the tank; whereby said pump circulates said volume of iron chelating liquid within said tank.

17. The method of claim 16, further comprising:
controlling the rate of circulation of said volume of iron chelating liquid.

18. The method of claim 16, further comprising:
controlling the speed of said pump with a variable frequency drive.

19. The method of claim 10, further comprising:
disposing said liquid contact tank on a pallet; and
disposing an enclosure on said pallet, said enclosure housing said tank.

20. The method of claim 19, further comprising:
disposing said pallet at a treatment facility; and
fluidly coupling a source of said gas containing sulfur compounds to said sparging device.
